(19) United States
(12) Patent Application Publication
(10) Pub. No.: US 2017/0245831 A1
(43) Pub. Date: Aug. 31, 2017

(54) Ultrasound Diagnostic Apparatus And Control Method Of Ultrasound Diagnostic Apparatus

(71) Applicant: KONICA MINOLTA, INC., Chiyoda-ku (JP)

(72) Inventors: Morio NISHIGAKI, Kanagawa (JP); Miyuki GAWAZAWA, Kanagawa (JP); Toshiharu SATO, Tokyo (JP); Yoshihiro TAKEDA, Tokyo (JP)

(73) Assignee: KONICA MINOLTA, INC., Tokyo (JP)

(21) Appl. No.: 15/437,813

(22) Filed: Feb. 21, 2017

(30) Foreign Application Priority Data
Feb. 26, 2016 (JP) 2016-035842

(51) Int. Cl. A61B 8/14; A61B 8/08; A61B 8/00

(52) U.S. Cl. CPC A61B 8/14; A61B 8/4483; A61B 8/54; A61B 8/0841; A61B 8/461; A61B 8/52

(57) ABSTRACT

Ultrasound diagnostic apparatus that receives a reception signal representing the reflection waves from ultrasound probe, includes position specifying section that specifies the depth of detection object inside the subject based on the temporal variation of the reception signal, and position specifying section that specifies the position of detection object in the first direction based on the reception signal generated based on the reflection waves from detection object obtained with the ultrasound beams transmitted at a first angle and a second angle which are different from each other in a transmission direction of the ultrasound beam.

### CROSS REFERENCE TO RELATED APPLICATIONS

This application is entitled to and claims the benefit of Japanese Patent Application No. 2016-035842, filed on Feb. 26, 2016, the disclosure of which including the specification, drawings and abstract is incorporated herein by reference in its entirety.

### BACKGROUND OF THE INVENTION

### 1. Field of the Invention

The present invention relates to an ultrasound diagnostic apparatus, and a control method of the ultrasound diagnostic apparatus.

### 2. Description of Related Art

Conventionally, an ultrasound diagnostic apparatus is known in which the inner side of a subject is inspected by transmitting an ultrasound beam into the subject, receiving the reflection waves (echo) thereof, and performing a predetermined signal data process. Such an ultrasound diagnostic apparatus is widely used in inspections and treatments for medical purposes.

In addition to the operation of displaying the image by processing the data of the acquired reflection waves, an ultrasound diagnostic apparatus generates an ultrasound image obtained by transmission and reception of ultrasound beams (hereinafter referred to as “ultrasound image”) at the time of collecting a sample at a specific portion (target) in the subject, discharging water or the like, or injecting and placing a medicine or a marker at a specific portion when the puncture needle is inserted to the target position while visually recognizing the puncture needle and the target position used for the above-mentioned operations, for example (ultrasound-guided paracentesis) (which will be described later see FIG. 6). With use of such an ultrasound image, the treatment on the target in the subject can be quickly, surely and readily performed.

Ultrasound diagnostic apparatuses in which transducers for transmission and reception of the ultrasound beam are one dimensionally disposed and are electrically switched for scanning (electronic scanning) to display a two-dimensional image are widely used. In ultrasound diagnostic apparatuses that display such an image, for example, when a puncture needle is inserted in the scanning direction (longitudinal axis direction), the puncture needle is located in a range where continuous imaging can be performed in a period until the puncture needle reaches the target from the insertion position of the subject. However, due to reflection from body tissues other than reflection from the puncture needle in a subject, it is difficult to visually recognize the puncture needle in an image including both of the image of the body tissue and the image of the puncture needle. In addition, in some situation, the puncture needle does not correctly advance along the insertion direction, or the puncture needle is bent due to the internal state of the subject, the structure, the end shape of the puncture needle and the like. As a result, the puncture needle is shifted in the direction orthogonal to the longitudinal axis direction (minor axis direction), and falls outside the imaging range, resulting in failure of the imaging of the puncture needle (or a part of the needle).

Some approaches have been made to solve the above-mentioned problems. For example, PTL 1 (Japanese Patent Application Laid-Open No. 2014-212922) discloses a technique of discriminating the puncture needle from the body tissue in which ultrasound image data corresponding to a plurality of frames is acquired, and the position of the reflector is specified for each frame to discriminate the puncture needle from the body tissue and detect the movement of the puncture needle. In addition, regarding the shifting of the position of the puncture needle in the minor axis direction, for example, PTL 2 (Japanese Patent Application Laid-Open No. 2000-139926) discloses a technique using a two-dimensional array transducer disposed in a direction orthogonal to the scanning direction in which a delay circuit for changing the operation timings is provided, and the levels of the delay amount of the plurality of transducers are switched to deflect the travelling direction of the ultrasound waves, thereby performing imaging of the outside of the normal transmission/reception width of the ultrasound waves.

In PTL 1, however, a plurality of pieces of the ultrasound image data are used to detect the movement of the puncture needle and specify the puncture needle. With such a configuration, reflection waves from the body tissue are also detected at the time of generation of an ultrasound image, and discrimination of the reflection waves of the body tissue from the reflection waves of the puncture needle cannot be performed, and consequently, the body tissue may erroneously detected as a puncture needle.

On the other hand, as disclosed in PTL 2, an electron delay part is required when an ultrasound beam is deflected by electronic scanning, and the amount of materials and the cost are increased.

On the other hand, there is a demand for detecting a puncture needle in an ultrasound tomogram, and for correctly specifying the amount of the shift of the puncture needle in the minor axis direction by deflecting an ultrasound beam in the minor axis direction.

### SUMMARY OF THE INVENTION

An object of the present invention is to provide an ultrasound diagnostic apparatus and a control method of the ultrasound diagnostic apparatus which can correctly specify the position of a detection object (in particular, the shifting amount of the ultrasound beam from the transmission/reception direction) when a detection object in the subject (such as a puncture needle and a body tissue) is detected with use of an ultrasound probe.

To achieve the abovementioned object, an ultrasound diagnostic apparatus reflecting one aspect of the present invention receives a reception signal representing reflection waves from an ultrasound probe, the ultrasound probe including: a plurality of transducers disposed along a first direction and configured to transmit an ultrasound beam to a subject and receive reflection waves thereof, and a transducer switching section configured to control a driving signal to the transducers to deflect a transmission direction of the ultrasound beam to the first direction, the ultrasound diagnostic apparatus including: a first position specifying section configured to specify a depth of a detection object inside the subject based on a temporal variation of the reception signal; a second position specifying section configured to specify a position of the detection object in the first direction based on reception signals generated based on reflection waves from the detection object obtained with the ultrasound beams transmitted at a first angle and a second angle which are different from each other in a transmission direction of the ultrasound beam; and an output control section configured to output the position of the detection object in the first direction such that an operator of the ultrasound probe is allowed to identify the position of the detection object in the first direction.

To achieve the abovementioned object, in a control method for an ultrasound diagnostic apparatus that receives a reception signal representing reflection waves from an ultrasound probe reflecting one aspect of the present invention, the ultrasound probe includes: a plurality of transducers disposed along a first direction and configured to transmit an ultrasound beam to a subject and receive reflection waves thereof, and a transducer switching section configured to control a driving signal to the transducers to deflect a transmission direction of the ultrasound beam to the first direction, the method including: specifying a depth of the detection object inside the subject based on a temporal variation of the reception signal; specifying a position of the detection object in the first direction based on reception signals generated based on reflection waves from the detection object obtained with the ultrasound beams transmitted at a first angle and a second angle which are different from each other in a transmission direction of the ultrasound beam; and outputting the position of the detection object in the first direction such that an operator of the ultrasound probe is allowed to identify the position of the detection object in the first direction.

### BRIEF DESCRIPTION OF DRAWINGS

FIG. 1 illustrates an example of a general configuration of an ultrasound diagnostic apparatus according to a first embodiment;

FIG. 2 is a block diagram illustrating an example of an internal configuration of the ultrasound diagnostic apparatus according to the first embodiment;

FIG. 3 illustrates an example of a transducer array in an ultrasound probe according to the first embodiment;

FIG. 4 illustrates an example of a cross-sectional structure along the minor axis direction in the transducer array of the ultrasound probe according to the first embodiment;

FIG. 5 illustrates an example of a relationship between the driven transducer and the transmission/reception direction of the ultrasound beam according to the first embodiment;

FIG. 6 illustrates an example of the positional relationship between the puncture needle and the ultrasound probe according to the first embodiment;

FIG. 7A illustrates an example of an image displayed by the ultrasound diagnostic apparatus according to the first embodiment;

FIG. 7B illustrates an example of an image displayed by the ultrasound diagnostic apparatus according to the first embodiment;

FIG. 8 illustrates an example of a control procedure executed by a control section at the time of generation of the image displayed by the ultrasound diagnostic apparatus according to the first embodiment;

FIG. 9A illustrates an example of a transmission/reception directivity characteristic curve of the ultrasound beam in accordance with the deflection angle according to the first embodiment;

FIG. 9B illustrates an example of the transmission/reception directivity characteristic curve of the ultrasound beam in accordance with the deflection angle according to the first embodiment;

FIG. 10A illustrates an example of the transmission/reception directivity characteristic curve of the ultrasound beam in accordance with the depth according to the first embodiment;

FIG. 10B illustrates an example of the transmission/reception directivity characteristic curve of the ultrasound beam in accordance with the depth according to the first embodiment;

FIG. 10C illustrates an example of the transmission/reception directivity characteristic curve of the ultrasound beam in accordance with the depth according to the first embodiment;

FIG. 11 illustrates a determination method for a continuous state according to the first embodiment;

FIG. 12 is an explanatory view of a method of specifying the minor axis position of a puncture needle according to a modification of the first embodiment;

FIG. 13 is an explanatory view of a method of specifying the minor axis position of a puncture needle according to a second embodiment;

FIG. 14 illustrates an example of a control procedure executed by a control section at the time of generation of an image displayed by an ultrasound diagnostic apparatus according to the second embodiment;

FIG. 15 illustrates an example of a notification section for indicating the minor axis position of a puncture needle according to a third embodiment; and

FIG. 16 is a block diagram illustrating an example of an internal configuration of an ultrasound diagnostic apparatus according to other embodiments.

### DESCRIPTION OF THE PREFERRED EMBODIMENTS

Embodiments of the present invention will be described below with reference to the accompanying drawings.

### First Embodiment

In the present embodiment, an example of an ultrasound diagnostic apparatus that generates an ultrasound image of a B mode (Brightness Mode) will be described.

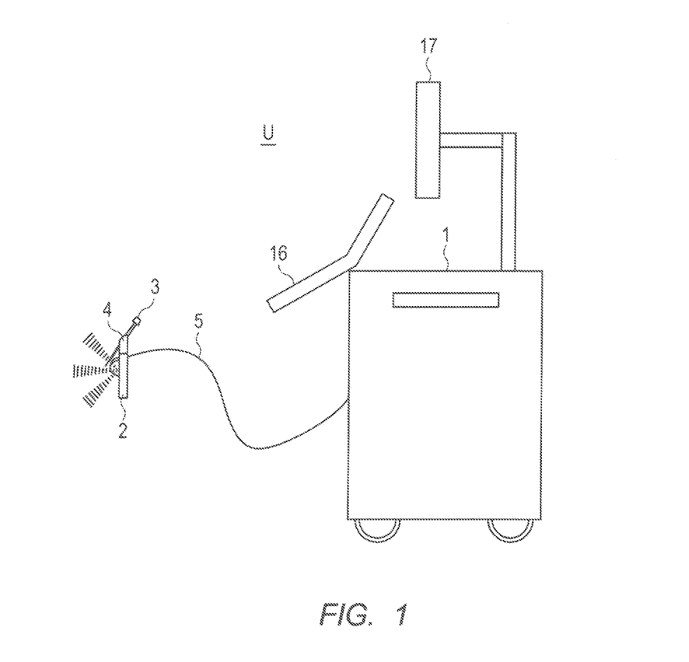

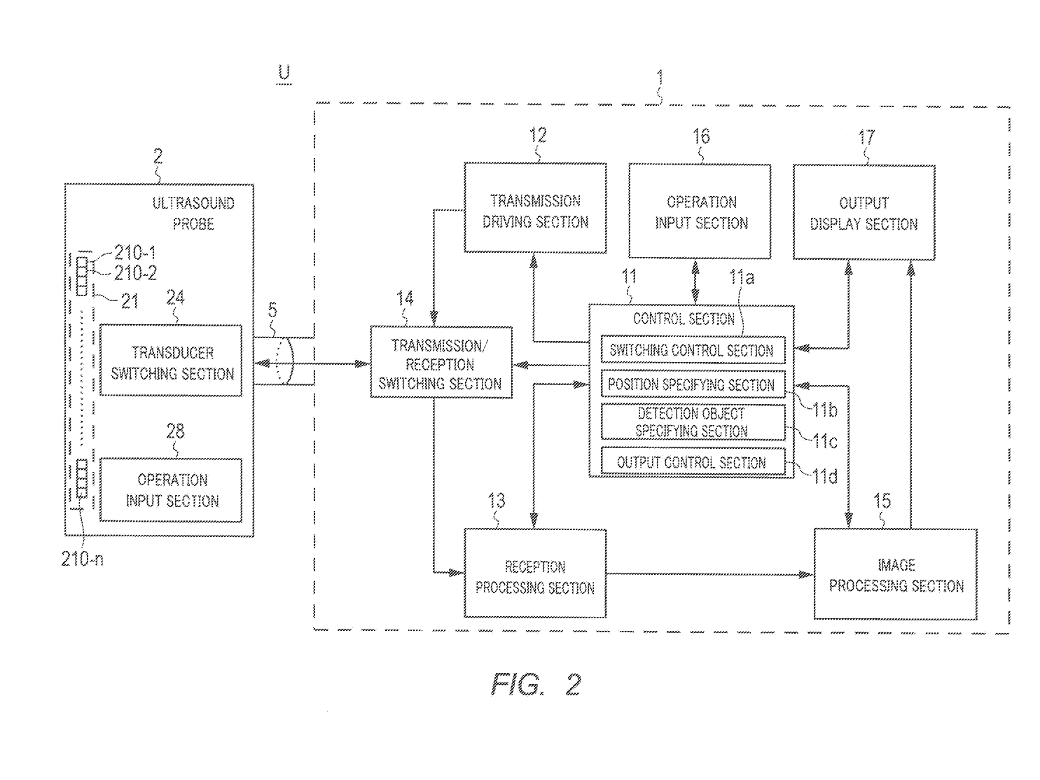

FIG. 1 illustrates an example of a general configuration of ultrasound diagnostic apparatus U according to the present embodiment. FIG. 2 is a block diagram illustrating an example of an internal configuration of ultrasound diagnostic apparatus U according to the present embodiment. Ultrasound diagnostic apparatus U includes ultrasound diagnostic apparatus main body 1, ultrasound probe 2 (ultrasound probe) connected with ultrasound diagnostic apparatus main body 1 through cable 5, puncture needle 3, attaching portion 4 attached to ultrasound probe 2, and the like.

Ultrasound probe 2 functions as an acoustic sensor that transmits an ultrasound beam (of about 1 to 30 MHz in this case) to a subject such as a living body, and receives reflection waves (echo) of the transmitted ultrasound waves reflected in the subject, and, converts the received reflection waves into an electric signal. Ultrasound probe 2 includes transducer array 21 that is an array of transducers 210 that transmit and receive an ultrasound beam, transducer switching section 24 that switches between transducers 210 for transmission and reception, operation input section 28, and the like.

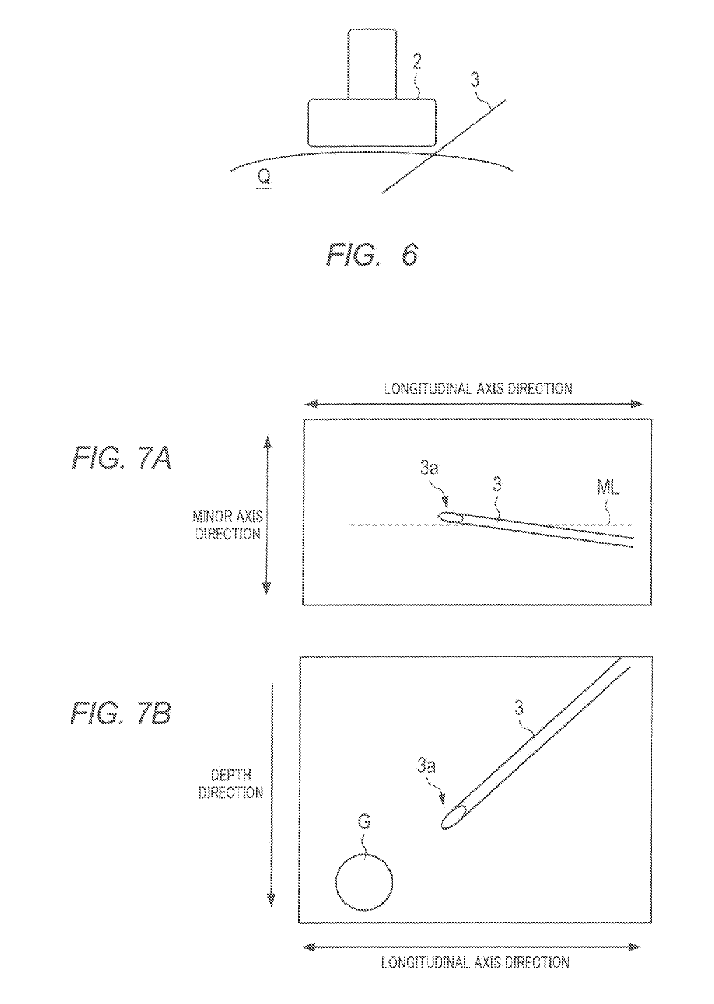

The operator brings the transmission/reception surface of the ultrasound beam of ultrasound probe 2, that is, the surface in the direction of transmission of ultrasound waves from transducer array 21, into contact with the subject, and operates ultrasound diagnostic apparatus U to perform ultrasound diagnosis (which will be described later with reference to FIG. 6). It is to be noted that, while ultrasound probe 2 transmits ultrasound waves into the subject from the outside (surface) and receives the reflection waves thereof in this case, ultrasound probe 2 may be an ultrasound probe which is designed for insertion to the digestive tract, the blood vessel or the like, or in the body cavity or the like, and may have any size and shape in accordance with the use.

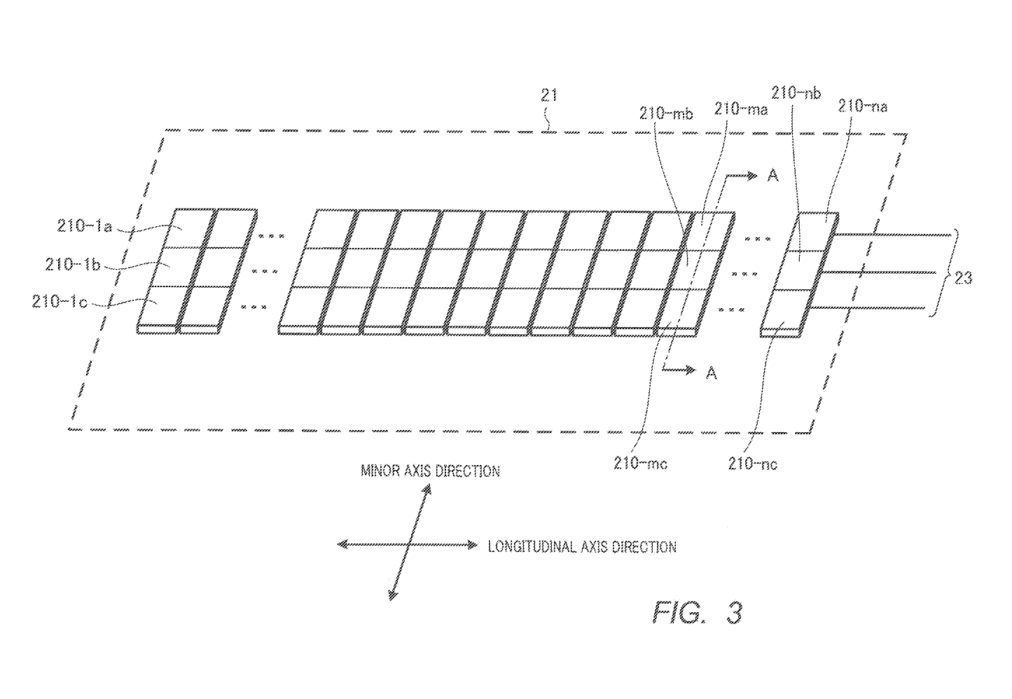

Transducer array 21 (which will be described later with reference to FIG. 3) is an array of a plurality of transducers 210. It is to be noted that transducer 210 is, for example, a piezoelectric element composed of a piezoelectric body and electrodes provided at both ends of the piezoelectric body.

Here, puncture needle 3 has a hollow long needle shape, and is inserted into the subject with an orientation set according to the setting of attaching portion 4. Puncture needle 3 can be exchanged with one having an appropriate thickness, length, and end shape in accordance with the types and amount of medicines to be injected, a collection target (sample) and the like.

Attaching portion 4 holds puncture needle 3 in a predetermined orientation (direction). Attaching portion 4 is attached on a side portion of ultrasound probe 2 such that the orientation of puncture needle 3 can be appropriately reset. Attaching portion 4 can not only simply move puncture needle 3 in the insertion direction, but also can insert puncture needle 3 while rotating (spinning) puncture needle 3 about the needle axis. It is to be noted that instead of providing attaching portion 4, it is also possible to directly provide ultrasound probe 2 with a guide part that holds puncture needle 3 in the insertion direction. Today, in some situation, puncturing is carried out without using attaching portion 4 depending on the object of the puncturing, the skill of the operator, and the like.

Ultrasound diagnostic apparatus main body 1 (hereinafter referred to also as “ultrasound diagnostic apparatus 1”) is provided with operation input section 16 and output display section 17. In addition, as illustrated in FIG. 2, ultrasound diagnostic apparatus main body 1 includes control section 11, transmission driving section 12, reception processing section 13, transmission/reception switching section 14, image processing section 15, and the like.

On the basis of an external inputting operation performed on the input device of operation input section 16 such as a keyboard and a mouse, control section 11 of ultrasound diagnostic apparatus main body 1 outputs a driving signal to ultrasound probe 2 to output an ultrasound beam. In addition, control section 11, acquires a reception signal representing the reflection waves from ultrasound probe 2, and carries out various processes, and, as necessary, displays a result and the like on the display screen of output display section 17 and the like.

Control section 11 includes switching control section 11a, position specifying section 11b, detection object specifying section 11c and output control section 11d.

Control section 11 is composed of a circuit of an arithmetic processor, a memory and the like. To be more specific, control section 11 includes a CPU (Central Processing Unit), a ROM (Read Only Memory), a RAM (Random Access Memory) and the like. The CPU reads various programs stored in the ROM, loads the programs in the RAM, and carries out centralized control of the operations of the components of ultrasound diagnostic apparatus U in accordance with the programs. The ROM stores various processing programs, setting data, control programs for operating ultrasound diagnostic apparatus U, and the like. Instead of the ROM, the above-mentioned programs and setting data may be stored in an auxiliary storage apparatus using a nonvolatile memory such as a flash memory including a SSD (Solid State Drive) in a readable and rewritable manner, for example. The RAM is a volatile memory such as a SRAM and a DRAM, provides the CPU with a work memory space, and stores temporarily data. The functions of switching control section 11a, position specifying section 11b, detection object specifying section 11c and output control section 11d are achieved when the CPU executes programs.

Switching control section 11a controls transmission driving section 12, reception processing section 13, and transmission/reception switching section 14 such that a driving signal is transmitted to a selected transducer of transducers 210-1 to 210-n, and a reception signal from a selected transducer of transducers 210-1 to 210-n is received. In addition, switching control section 11a controls transducer switching section 24 of ultrasound probe 2 to individually switch selected transducers for transmission and reception among transducers 210-1 to 210-n of transducer array 21 (which will be described later with reference to FIG. 3).

In addition, position specifying section 11b specifies the position of tip portion 3a of puncture needle 3 based on a reception signal acquired from reception processing section 13 (which will be described later with reference to FIG. 7). It is to be noted that position specifying section 11b includes a first position specifying section that specifies the depth of tip portion 3a of puncture needle 3, and a second position specifying section that specifies the position of tip portion 3a of puncture needle 3 in the minor axis direction.

In addition, detection object specifying section 11c specifies the type of the detection object (puncture needle, body tissue or the like) based on the continuous state of the minor axis position of the detection object in the longitudinal axis direction.

In addition, on the basis of the data representing the position and the type of the detection object specified by position specifying section 11b and detection object specifying section 11c, output control section 11d outputs the minor axis position of puncture needle 3 such that the operator of ultrasound probe 2 can identify the minor axis position of puncture needle 3. Here, output control section 11d outputs the minor axis position of puncture needle 3 to image processing section 15 to generate a planar image representing the position of puncture needle 3 and display the image on output display section 17.

Transmission driving section 12 is a circuit that outputs a pulse signal (hereinafter referred to also as “driving signal”) to be supplied to ultrasound probe 2 in accordance with the control signal input from control section 11 such that ultrasound probe 2 transmit ultrasound waves. The configuration and the operation of transmission driving section 12 are publicly known, and are not directly related with the subject application, and therefore, the description thereof will be omitted.

Reception processing section 13 is a circuit that acquires a reception signal input from ultrasound probe 2 under the control of control section 11. Reception processing section 13 has a gain variable amplifier, an A/D conversion circuit, and a phasing addition circuit, for example. The phasing addition circuit generates beam-formed sound ray data by adding (phasing addition) a delay time in accordance with the position of transducer 210 to an A/D converted reception signal. The configuration and the operation of reception processing section 13 are publicly known, and are not directly related with the subject application, and therefore, the description thereof will be omitted.

Transmission/reception switching section 14 is a circuit configured to operate such that, under the control of control section 11, a driving signal is transmitted from transmission driving section 12 to transducer 210 in the case where an ultrasound beam is transmitted from transducer 210, and to perform a switching operation for outputting a reception signal to reception processing section 13 in the case of acquiring a signal corresponding to the ultrasound waves transmitted from a selected transducer of transducers 210-1 to 210-n.

Image processing section 15 generates a diagnostic image based on the received data of the ultrasound waves. In addition, image processing section 15 acquires a signal by detecting (envelope detection) sound ray data input from reception processing section 13, and, as necessary, performs logarithmic amplification, filtering (such as low-pass and smoothing), emphasis processing, adjustment of the dynamic range and the like.

As one example of the diagnostic image, image processing section 15 generates each frame image (diagnosis image) data according to a B mode display representing a two-dimensional structure in a cross section including the transmission/reception direction of the signal (the depth direction of the subject) and the scanning direction of the ultrasound waves transmitted and received by ultrasound probe 2. In addition, image processing section 15 generates a planar image representing the position of tip portion 3a of puncture needle 3 on the basis of the data representing the type of the detection object and the data for specifying the position of tip portion 3a of puncture needle 3 received from control section 11 (position specifying section 11b and detection object specifying section 11c).

Image processing section 15 is composed of a circuit of an arithmetic processor, a memory and the like, and performs scan conversion of data of reception processing section 13, and image processing such as inter-frame processing of the image. To be more specific, a configuration including a dedicated CPU and a dedicated RAM used for the above-mentioned image generations may be adopted. In image processing section 15, a dedicated hardware configuration for image generation may be formed on a substrate (ASIC (Application-Specific Integrated Circuit) or the like), or may be formed with an FPGA (Field Programmable Gate Array). Alternatively, image processing section 15 may have a configuration in which image generation processing is performed with the CPU and the RAM of control section 11. Image processing section 15 includes a storage section, and stores diagnostic image data (frame image data) in the frame unit for an immediately preceding predetermined frame numbers. The diagnostic image data stored in the storage section is read under the control of control section 11 and transmitted to output display section 17 or output to the outside of ultrasound diagnostic apparatus U through a communication line (not illustrated). At this time, in the case where the display system of output display section 17 is a television system, it suffices that a DSC (Digital Signal Converter) is provided between the storage section and the output display section 17 and that the diagnostic image data stored in the storage section is output after the scan format is converted.

Operation input section 16 includes a push button switch, a keyboard, a mouse, a trackball, a touch panel on an output display section, or a combination thereof. Operation input section 16 converts the inputting operation of the operator into an operation signal and inputs the operation signal to ultrasound diagnostic apparatus main body 1.

Output display section 17 includes a display screen of various types such as an LCD (Liquid Crystal Display) and the driving section thereof. In accordance with a control signal output from control section 11 and image data output from image processing section 15, output display section 17 generates a display screen (each display pixel) and indicates a menu and a status according to the ultrasound diagnosis, and measurement data based on the received ultrasound waves on the display screen.

Cable 5 includes a connector (not illustrated) for ultrasound diagnostic apparatus main body 1 at one end thereof, and with the connector, ultrasound probe 2 is detachably attached to ultrasound diagnostic apparatus main body 1.

FIG. 3 illustrates an example of transducer array 21 of ultrasound probe 2 according to the present embodiment. Transducer array 21 in ultrasound diagnostic apparatus U according to the present embodiment is a plurality of transducers 210-1a to c, 210-2a to c, . . . , 210-na to c which are disposed in a matrix in a two-dimensional surface (which may not be a plane) defined with a predetermined direction (longitudinal axis direction, or second direction) and the minor axis direction (first direction) orthogonal to the longitudinal axis direction. Here, the number of transducers 21 in the longitudinal axis direction is greater than the number of transducers 21 in the minor axis direction. In the minor axis direction, three transducers (for example, at the left end of FIG. 3, 210-1a, 210-1b, and 210-1c) are sequentially disposed. The group of three transducers 210a to 210c disposed along the minor axis direction is hereinafter referred to also as “transducer group.” Each of transducers 210-1a to c, 210-2a to c, 210-na to c of transducer array 21 is connected with line path 23 in cable 5, and can be individually supplied with a voltage pulse with a switching device and the like. In other words, each of transducers 210-1a to c, 210-2a to c, . . . , 210-na to c of transducer array 21 can be individually switched between the driving state and non-driving state.

When the drive pulse is supplied to transducers 210-1a to c, 210-2a to c, . . . , 210-na to c and piezoelectric bodies of transducers 210-1a to c, 210-2a to c, . . . , 210-na to c supplied with the drive pulse are deformed (expanded and contracted) in accordance with the electric field, an ultrasound beam is transmitted. At this time, an ultrasound beam is transmitted to a position and a direction in accordance with the position and the direction of transducers 210-1a to c, 210-2a to c, . . . , 210-na to c supplied with the voltage pulse, the converging direction of the ultrasound beam, and the degree of the shift (delay) of the timing. In addition, when reflection waves reflected in the subject are incident on transducers 210-1a to c, 210-2a to c, . . . , 210-na to c, the thickness of the piezoelectric body is varied (vibrated) due to the sound pressure of the incidence and an electric charge in accordance with the amount of variation is generated, which is converted into an electric signal in accordance with the amount of the electric charge, and output as a reception signal to reception processing section 13. When a B mode image is generated (which will be described later with reference to FIG. 7), a drive pulse is sequentially supplied along the longitudinal axis direction in a unit of a predetermined number of transducer groups (which may include partial overlap).

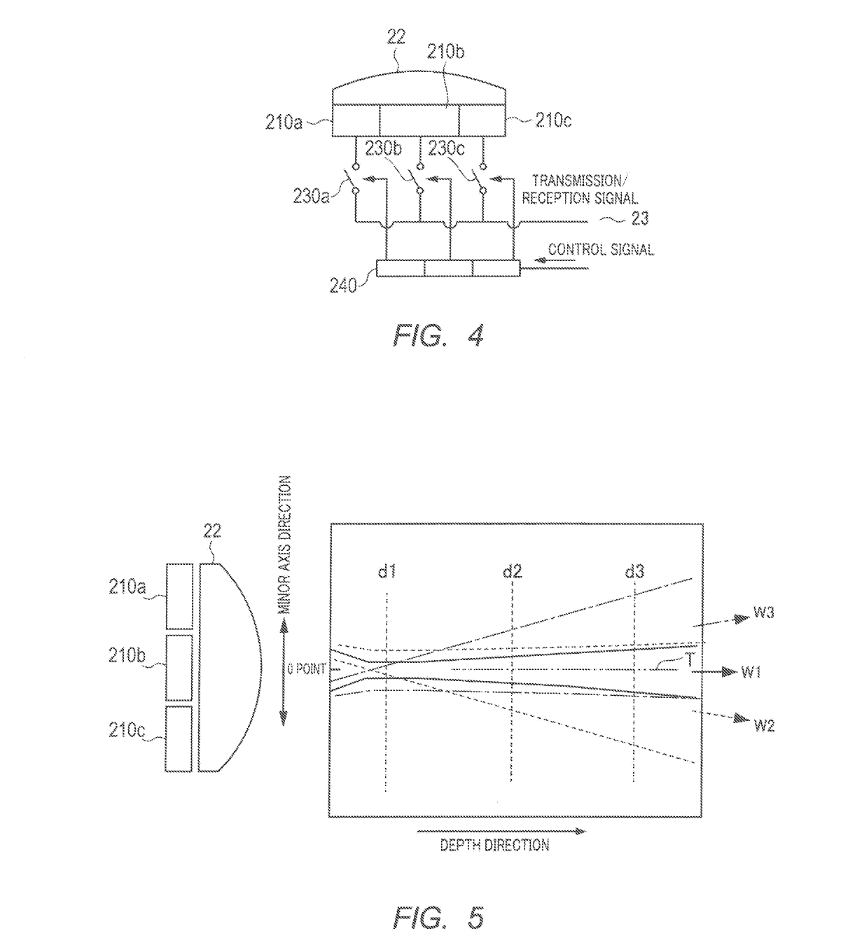

Transducer switching section 24 is a selector for switching the driving state of each of transducers 210-1a to c, 210-2a to c, . . . , 210-na to c of transducer array 21. Transducer switching section 24 includes a switching device (which will be described later with reference to FIG. 4) for switching between the driving state and the non-driving state of each of transducers 210-1a to c, 210-2a to c, . . . , 210-na to c in the minor axis direction. While transducer switching section 24 is configured to switch between the driving state and the non-driving state of the transducers a to c of the transducer groups, transducer switching section 24 may also be used for the purpose of switching the opening position in the longitudinal axis direction, in principle.

Operation input section 28 of FIG. 2 receives the inputting operation of the operator and operates such that an operation in accordance with the input operation is performed. For example, the setting of transducer switching section 24 can be changed by manually operating operation input section 28.

Next, a configuration for deflection of the transmission/reception direction of the ultrasound beam in ultrasound diagnostic apparatus U of the present embodiment will be described.

FIG. 4 illustrates a cross-sectional structure of transducer array 21 of ultrasound probe 2 of the present embodiment along the minor axis direction (transducer group). Here, FIG. 4 illustrates a cross-sectional structure of transducer groups 210-ma to 210-mc taken along the minor axis cross-section A-A of FIG. 3.

As illustrated in FIG. 4, in ultrasound probe 2, convex acoustic lens 22 (converge section) is provided to three transducers 210a to 210c disposed in the minor axis direction. With this configuration, an ultrasound beam generated by transducers 210a to 210c is refracted by acoustic lens 22, and the transmission/reception width of the beam converges at the center point in the minor axis direction. Normally, silicone or the like is used for acoustic lens 22. Alternatively, other materials may be appropriately selected in accordance with a desired ultrasound refractive index.

In addition, ultrasound probe 2 is provided with switching devices 230a to 230c for controlling the on/off of each transducer 210 in the minor axis direction, and register 240 (corresponding to transducer switching section 24). Switching devices 230a to 230c, which are respectively interposed between line path 23 and transducers 210a to 210c, control the output of a driving signal to transducers 210a to 210c, and control acquisition of reception signals generated by transducers 210a to 210c. While the type of switching devices 230a to 230c is not limited, it is preferable to use an FET (field effect transistor) in terms of the power consumption amount, the pressure resistance under the transmission and reception of ultrasound waves, and the like, for example.

In addition, each of the control electrodes of switching devices 230a to 230c are connected with register 240, and switching devices 230a to 230c perform switching between on and off based on the switching signal output from register 240. Register 240 stores a setting for deflecting the transmission/reception direction of the ultrasound beam in the minor axis direction, and, in accordance with the setting, outputs a switching signal to the control electrodes of switching devices 230a to 230c to perform switching between on and off of switching devices 230a to 230c. It is to be noted that a control signal is serially transmitted to register 240 from control section 11 (switching control section 11a) to change the setting. In this manner, the setting of register 240 can be changed with a serial signal, and thus the number of signal lines between control section 11 and register 240 is reduced.

In this manner, transducer switching section 24 changes the combination to be switched between on and off in the transducer group of switching devices 230a to 230c to thereby deflect the transmission/reception direction of the ultrasound beam in the minor axis direction by the transducer group. With this configuration, transducer switching section 24 can set register 240 for each transducer group disposed along the longitudinal axis direction. In other words, transducer switching section 24 can set the deflection angle of the transmission/reception direction of the ultrasound beam for each transducer group. It is to be noted that, in the case where switching device 230 is turned off, no driving signal is sent to corresponding to transducer 210, and consequently no reception signal generated by reflection waves is output from that transducer 210. It should be noted that transducer switching section 24 may be controlled such that only the transmission of the ultrasound beam is turned off whereas the reception of the reflection waves is performed.

FIG. 5 illustrates an example of the relationship between driven transducers 210a to 210c and the transmission/reception direction of the ultrasound beam. In FIG. 5, the ordinate and the abscissa correspond to the minor axis direction and the depth direction, respectively, and transmission directions of ultrasound beams from the center point 0 of the transmission/reception surfaces of transducers 210a to 210c are illustrated. On the left side of the graph of FIG. 5, the positions of transducers 210a to 210c are illustrated along the ordinate. It is to be noted that the direction in which transducers 210a to 210c receive the reflection waves is opposite to the transmission direction of the ultrasound beam.

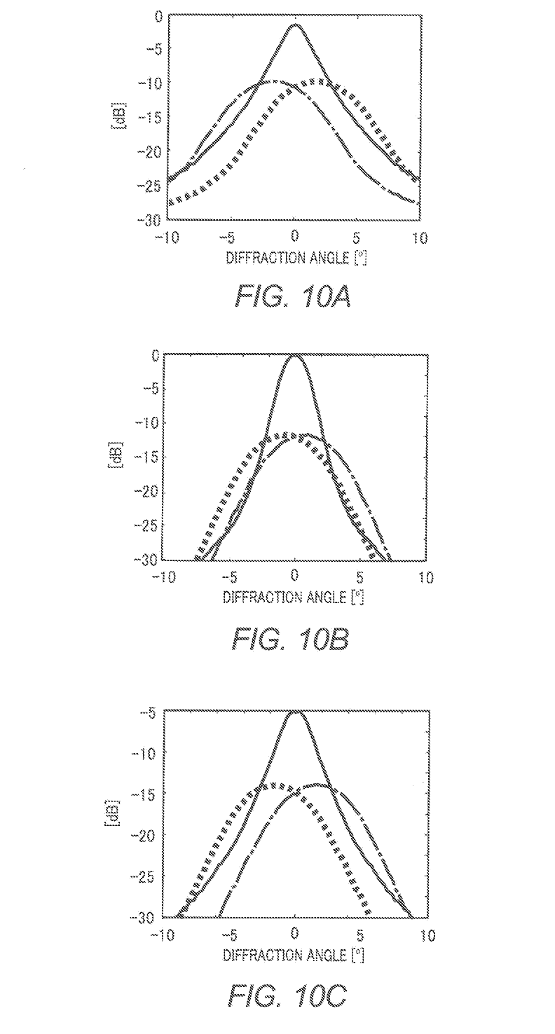

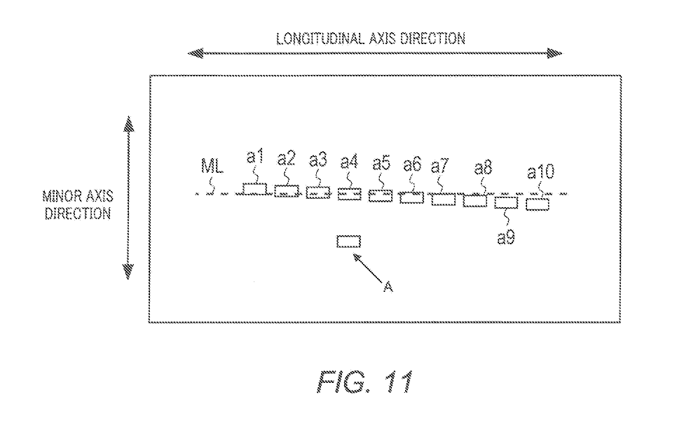

Here, solid line W1 indicates a transmission direction of an ultrasound beam in the case where all transducers 210a to 210c are driven. Dotted line W2 indicates a transmission direction of an ultrasound beam in the case where only transducer 210a is driven. Dashed line W3 indicates a transmission direction of an ultrasound beam in the case where only transducer 210c is driven. Solid line W1, dotted line W2, and dashed line W3 indicate the positions of −6 dB with respect to the peak value of the intensity of the ultrasound transmission/reception beam at each depth, and the maximum point of the directivity at each depth is present between the two lines. In other words, the directivity of an ultrasound beam varies depending on the depth. Therefore, the widths between two solid lines W1, two dotted lines W2, and two dashed lines W3 (positions of −6 dB with respect to the peak value of the signal intensity of the ultrasound beam) differ depending on depths d1, d2 and d3 in FIG. 5 (which will be described later with reference to FIG. 10).

As illustrated in FIG. 5, in the case where all transducers 210a to 210c are used (solid line W1), the ultrasound beam rectilinearly travels without being deflected (at a deflection angle of 0 degree, which is hereinafter referred to also as “first ultrasound beam”). On the other hand, in the case where only transducer 210a is used (dotted line W2), the transmission direction of the ultrasound beam is deflected to transducer 210c side in the minor axis direction with respect to the center point 0 (at a deflection angle of substantially −3 degrees, which is hereinafter referred to also as “second ultrasound beam”) with refraction of acoustic lens 22. In addition, in the case where only transducer 210c is used (dashed line W3), the transmission direction of the ultrasound beam is deflected to transducer 210a side in the minor axis direction with respect to the center point 0 (at a deflection angle of substantially +3 degrees, which is hereinafter referred to also as “third ultrasound beam”). It is to be noted that, in the drawing, the chain double-dashed line (line T) indicates the central axis of the ultrasound beam. In addition, the deflection angle is the angle to the central axis line T of the ultrasound beam, or in other words, the angle to the minor axis direction changed from the normal direction of the transmission/reception surface of the ultrasound beam of transducers 210a to 210c (hereinafter referred to also as “deflection angle of the ultrasound beam”). In addition, the depth direction is the internal direction of subject Q (FIG. 6) in which the ultrasound beam is transmitted, and the depth is the internal depth position of subject Q and the distance from the transmission/reception surface of the ultrasound beam of transducers 210a to 210c.

In this manner, by use of a part of transducers 210a to 210c in the minor axis direction, the transmission/reception direction of the ultrasound beam in the minor axis direction can be deflected. In addition, in the case where transducers 210b and 210c are driven without driving transducer 210a, the deflection angle to the minor axis direction can be further reduced in comparison with the case where only transducer 210c is driven. It is possible to further minutely change the deflection angle by controlling the driving state and the non-driving state of transducer 210 in the longitudinal axis direction (for example, transducers 210 of the line corresponding to transducer 210c in the longitudinal axis direction are alternately turned on and off, and the even-numbered transducers in the longitudinal axis direction are turned on and used for transmission and reception, and, the odd-numbered transducers in the longitudinal axis direction are turned off and are not used for transmission and reception) at the time of selecting the transducer 210 to be driven.

### <Method of Generating Ultrasound Image>

Next, a method of generating an ultrasound image according to the present embodiment will be described with reference to FIGS. 6 to 11.

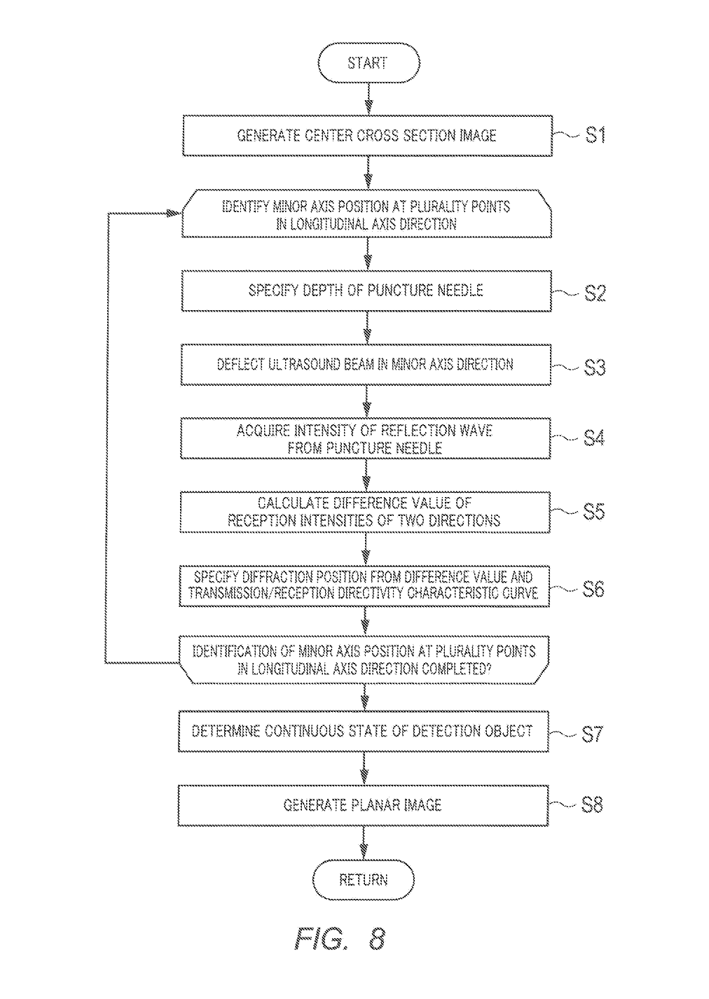

FIG. 6 illustrates an example of a positional relationship between ultrasound probe 2 and puncture needle 3. FIG. 7A and FIG. 7B illustrate an example of an image displayed by ultrasound diagnostic apparatus U. FIG. 8 illustrates an example of a control procedure of control section 11 at the time of generating the image displayed by ultrasound diagnostic apparatus U.

In the present embodiment, an aspect of inserting puncture needle 3 into the subject Q by a parallel method is described (see FIG. 6). By the parallel method, puncture needle 3 is inserted into the inside of the skin of the subject Q from the outer skin portion of the subject Q along the longitudinal axis direction of ultrasound probe 2.

In the case where puncture needle 3 is inserted toward target G (for example, tumor) inside the skin of the subject Q, ultrasound diagnostic apparatus U according to the present embodiment displays an ultrasound tomographic image (FIG. 7B) and an image (FIG. 7A) of puncture needle 3 in plan view. These images are generated by transmitting an ultrasound beam toward the inside of the subject, and by receiving the reflection waves thereof with ultrasound probe 2 making contact with the surface of the subject Q.

The ultrasound tomographic image illustrated in FIG. 7B is a B mode image. The B mode image is generated when an ultrasound beam is transmitted toward the inside (substantially in a direction of the normal of the transmission/reception surface of transducer 210) of the subject Q based on the temporal variation of the reception signal of the reflection waves. In other words, the B mode image is generated based on the time period until the transmitted ultrasound waves are reflected and returned and the intensity thereof, as an internal tomogram of the subject Q. The B mode image is a tomographic image of a cross section with the longitudinal axis direction of ultrasound probe 2 and the internal direction of the tissue, and shows the user the positional relationship between puncture needle 3 (in particular, tip portion 3a) and target G

The planar image illustrated in FIG. 7A is generated as an image illustrating the position of puncture needle 3 in the minor axis direction specified at a plurality of points in the longitudinal axis direction (hereinafter referred to as “minor axis position”). This planar image is generated as an image of puncture needle 3 in plan view at a position corresponding to the longitudinal axis direction of the B mode image so that the operator grasps the minor axis position of puncture needle 3. It is to be noted that center line ML of FIG. 7A indicates the position of the central axis of the ultrasound beam, which is the position of the center cross-section of the transmission/reception surface in the minor axis direction in the case where transducers 210a to 210c are not deflected. FIG. 7A may not be an ultrasound B mode tomogram as illustrated in FIG. 7B as long as the position of the puncture needle in the minor axis direction and the longitudinal axis direction can be determined. Practically, it suffices to indicate a mark or the like of the puncture needle.

First, a procedure for determining the minor axis position of puncture needle 3 at a certain point will be described.

First, control section 11 (position specifying section 11b) specifies the depth of puncture needle 3 at the point. Since puncture needle 3 has a high ultrasound reflection intensity (the difference of the acoustic impedance with respect to the internal cells of the human bodies is large), the depth of puncture needle 3 is determined as a timing when the signal intensity increases during detection of the temporal variation of the reception signal of the reflection waves. Therefore, the depth of puncture needle 3 is determined at the time of generating the B mode image. It is to be noted that the depth of puncture needle 3 is the distance between the ultrasound beam transmission/reception surface of transducer 210 and the portion of puncture needle 3 where the reflection waves are generated.

By specifying the depth of puncture needle 3, the transmission/reception directivity characteristic curve of the ultrasound beam which is referred to at the time when specifying the minor axis position of puncture needle 3 can be specified. It is to be noted that the transmission/reception directivity characteristic curve of the ultrasound beam is different depending on the depth of puncture needle 3 (which will be described later with reference to FIG. 10).

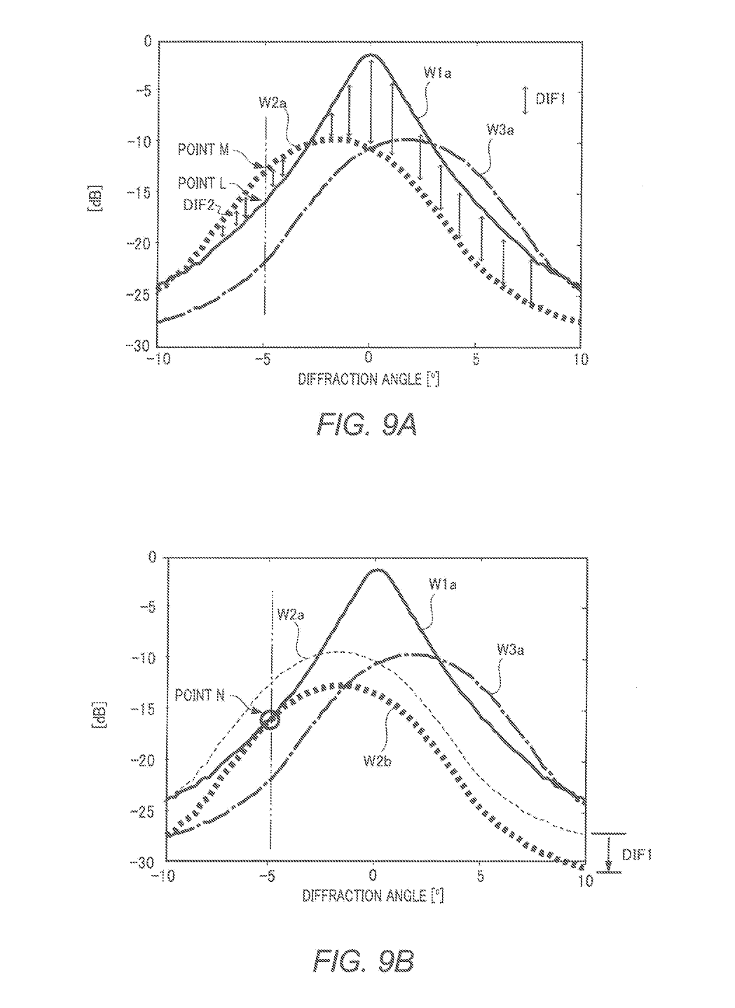

FIG. 9A illustrates an example of the transmission/reception directivity characteristic curve of the ultrasound beam.

The transmission/reception directivity characteristic curve is data representing the relationship between the signal intensity of a reception signal and the position of the detection object in the minor axis direction which is assumed to be obtained in the case where an ultrasound beam is transmitted and received with respect to the detection object under a predetermined measurement condition (hereinafter referred to also as “transmission/reception directivity characteristic data”). The transmission/reception directivity characteristic curve can be experimentally determined for each point, or can be determined by a simulation based on the distances between puncture needle 3 and the transmission/reception points of the ultrasound beam, the acoustic impedance of puncture needle 3 and the subject tissue Q, the attenuation characteristics of the ultrasound waves in the subject Q, and the like. Examples of the predetermined measurement condition include the transducer to be driven, the distance between the transducer to be driven and the detection object, the deflection angle of the ultrasound beam, the transmission intensity of the ultrasound beam, the pulse width, the attenuation rate in the subject, and the like.

FIG. 9A corresponds to FIG. 5 and illustrates transmission/reception directivity characteristic curves W1a (of a first ultrasound beam having a deflection angle of 0 degrees), W2a (of a second ultrasound beam having a deflection angle of −3 degrees), and W3a (of a third ultrasound beam having a deflection angle of +3 degrees) at a certain depth in the case where the ultrasound beam is deflected. The ordinate (dB) indicates a transmission function (of the reception intensity of the reflection waves from puncture needle 3/the signal intensity of the transmission signal of the ultrasound beam). In other words, the ordinate (dB) indicates a signal intensity (hereinafter referred to also as “reception intensity”) of an ultrasound beam which is reflected by puncture needle 3 and detected when it is assumed that puncture needle 3 is virtually present at a deflection angle the abscissa (minor axis position) at the depth. In addition, the abscissa (degree) indicates the deflection angle with respect to the minor axis direction with a depth set as a reference in the depth direction (the normal direction of the transmission/reception surface of ultrasound probe 2). With reference to the transmission/reception directivity characteristic curve illustrated in FIG. 9A, it can be said that the orientation directions are different among the first ultrasound beam (solid line W1a), the second ultrasound beam (dotted line W2a), and the third ultrasound beam (dashed line W3a), and that the peaks of the second and third ultrasound beams are relatively lower than the peak of the first ultrasound beam by approximately 7 dB, for example.

In general, the minor axis position of puncture needle 3 or the like can be specified from the deflection angle at the time when the signal intensity of a reception signal is maximized when the transmission/reception direction of the ultrasound beam is deflected. In view of this, in the case where transducer 210 which is used in transmission and reception in the transducer group in the minor axis direction is switched and the transmission/reception direction of the ultrasound beam is deflected with respect to the minor axis direction, adjustment of the deflection angle is stepwise, and therefore the minor axis position of puncture needle 3 is specified by referring to the above-mentioned transmission/reception directivity characteristic curve and the deflection angle at the time when the signal intensity of a reception signal has a high value to a certain degree.

However, as the transmission/reception directivity characteristic curve of FIG. 9A indicates, when the deflection angle of the ultrasound beam is changed by the switching of the transducers (dotted line, dashed line), the directivity of the ultrasound beam is wide, and the degree of the variation of the reception intensity of puncture needle 3 is small around the peak. As a result, when specifying the minor axis position of puncture needle 3, the error range is wide (for example, about −8 degrees to −2 degrees), and consequently the minor axis position of puncture needle 3 cannot be correctly specified. In addition, when the transducer which is used in transmission and reception is selected with use of the switch, the variation of the deflection angle is small, and the deflection specifically directed toward the position of the puncture needle cannot be achieved.

In view of this, in ultrasound diagnostic apparatus U according to the present embodiment, the transmission/reception direction of the ultrasound beam is deflected, and the reception signals of the deflection angles of the two directions (including 0 degree) are detected such that the errors due to the above-mentioned gentle directivity and the limited options of the deflection angle are offset with use of the reception signals of the two directions, and consequently the present embodiment could achieve improvement of the resolution. To be more specific, as illustrated in FIG. 9B, ultrasound diagnostic apparatus U according to the present embodiment uses a difference value DIF1 between the reception intensity of the reflection waves from puncture needle 3 when a first ultrasound beam at a deflection angle in the minor axis direction is transmitted and received, and the reception intensity of the reflection waves from puncture needle 3 when a second ultrasound beam at another deflection angle is transmitted and received. FIG. 9B illustrates an example of a method of searching the position where the difference value DIF1 between the reception intensity obtained using the first ultrasound beam and the reception intensity obtained using the second ultrasound beam is identical with the difference value DIF2 between transmission/reception directivity characteristic curve W2a and transmission/reception directivity characteristic curve W1a. In this method, the reflectance of the puncture needle is substantially constant.

FIG. 9B is an explanatory view of a method of specifying the minor axis position of puncture needle 3 according to the present embodiment. FIG. 9B corresponds to FIG. 9A and is additionally includes line W2b lowered from second the transmission/reception directivity characteristic curve of the ultrasound beam W2a by 3 dB as a whole. Here, for convenience of description, it is assumed that the detected reception intensity with use of the first ultrasound beam is −16 dB and that the detected reception intensity with use of the second ultrasound beam is −13 dB; in other words, the difference value DIF1 is 3 dB.

To be more specific, first, control section 11 (position specifying section 11b) obtains a difference value DIF1 of 3 dB from the reception intensity of −16 dB from puncture needle 3 detected with use of the first ultrasound beam, and the reception intensity of −13 dB from puncture needle 3 detected with use of the second ultrasound beam. Next, control section 11 (position specifying section 11b) shifts the original transmission/reception directivity characteristic curve of the second ultrasound beam (dotted line W2a) as a whole by the difference value DIF1 of 3 dB to generate dotted line W2b. In this manner, control section 11 (position specifying section 11b) can specify that the same difference value DIF2 3 dB between the reception intensity obtained using the first ultrasound beam and the reception intensity obtained using the second ultrasound beam can be obtained at the intersection N between the dotted line W2b and first transmission/reception directivity characteristic curve W1a of the ultrasound beam. In other words, control section 11 (position specifying section 11b) can specify that the minor axis position of puncture needle 3 is at the position of intersection N (−5 degrees).

In this manner, in the present embodiment, the minor axis position of puncture needle 3 is specified with use of the transmission/reception directivity characteristic curve based on the reception intensity of the reflection waves from puncture needle 3, and the transmission/reception directivity characteristic curves corresponding to the difference of the reception intensities of the two directions, in place of the method directly specifying the minor axis position of puncture needle 3. In this manner, the errors due to the above-mentioned gentle directivity and the limited options of the deflection angle can be offset. In other words, the resolution of the measurement of the minor axis position of puncture needle 3 can be improved. It is to be noted that control section 11 (position specifying section 11b) may determine the position of the detection object in the minor axis direction as coordinate data, or a deflection angle.

In the above-mentioned manner, the minor axis position of puncture needle 3 at a certain point can be determined.

It is to be noted that when ultrasound beams of the two directions are used, it is desirable to use deflection angles which cause less error. As can be seen in FIG. 9A and FIG. 9B, for example, when transmission/reception directivity characteristic curves W1a and W3a are used in the case where the determination is performed at a deflection angle of around −3 degrees, the inclinations of the curves are almost identical to each other around −3 degrees, and therefore the error increases when the above-mentioned method is used. In this case, by using transmission/reception directivity characteristic curve W2a in place of transmission/reception directivity characteristic curve W3a, highly accurate results can be obtained.

FIG. 10A, FIG. 10B, and FIG. 10C illustrate examples of the transmission/reception directivity characteristic curve of the ultrasound beam in accordance with the depth. While the transmission/reception directivity characteristic curves at a certain depth are illustrated in FIG. 9A and FIG. 9B, the directivity (reception intensity) of the ultrasound beams differs depending on the depth. Therefore, the transmission/reception directivity characteristic curve differs depending on the depth even when the same deflection angle of the transmission/reception direction of the ultrasound beam is used.

FIG. 10A, FIG. 10B, and FIG. 10C illustrate the transmission/reception directivity characteristic curves of the ultrasound beams at positions corresponding to depths d1, d2, and d3 illustrated in FIG. 5, respectively. Each of FIG. 10A, FIG. 10B, and FIG. 10C corresponds to FIG. 5 and illustrates transmission/reception directivity characteristic curves of a first ultrasound beam (solid line) of the case where all transducers of the transducer group are used, a second ultrasound beam (dotted line) of the case where only transducer 210a is used, and a third ultrasound beam (dashed line) of the case where only transducer 210c is used. By determining the transmission/reception directivity characteristic curve for each depth in advance in this manner, the minor axis position of puncture needle 3 can be determined with use of the corresponding transmission/reception directivity characteristic curve in accordance with the depth where puncture needle 3 is detected. In addition, as can be seen from the transmission/reception directivity characteristic curves of FIG. 10A and FIG. 10B, and FIG. 10C, at a near position with respect to the focus of the transducer lens, the beam position in the minor axis direction is reversed between left and right, and therefore it is impossible to simply determine the left side ultrasound beam and the right side ultrasound beam. Also in view of this, the determination of the correct position in the above-mentioned manner is meaningful.

Next, with reference to FIG. 8, a process for generating a planar image (FIG. 7A) is described. Here, a process for generating a planar image (FIG. 7A) is performed after a process for generating a B mode image (FIG. 7B) is performed under the control of control section 11.

When an image display process is started, control section 11 controls image processing section 15 to generate a B mode image of a center cross-section (S1). To be more specific, control section 11 (switching control section 11a) controls transducer switching section 24 to sequentially switch transducer 210 to be driven in transducer array 21 along the longitudinal axis direction so that a B mode image is generated (array type electronic scanning)

At this time, transducer 210 transmits a pulsed ultrasound beam in the depth direction, and, after the transmission of the ultrasound beam, receives the reflection waves from a reflector (such as target G and puncture needle 3). Then, transducer 210 generates a reception signal having a signal intensity in accordance with the sound pressure of the reflection waves, and transmits the reception signal to reception processing section 13. The reception signal generated by transducer 210 is subjected to A/D conversion or the like at reception processing section 13, and thereafter stored in the line memory of image processing section 15 as the temporal variation of the signal intensity (amplitude). Then, transducer 210 for transmission and reception of the ultrasound beam is switched along the longitudinal axis direction sequentially and individually or in a block unit (in a unit of a plurality of transducers 210). In this manner, the reception signal is stored in a plurality of line memories along the longitudinal axis direction, and the signal intensity of the reception signal is converted into a luminance, whereby a two-dimensional B mode image is generated.

A planar image (FIG. 7A) is generated by repeating the steps of (S2) to (S6), and specifying the minor axis position of puncture needle 3 in the minor axis direction at a plurality of points in the longitudinal axis direction.

First, control section 11 (position specifying section 11b) specifies the depth of the object point where the minor axis position of puncture needle 3 is determined based on the temporal variation of the reception signal at the time when the B mode image is acquired (S2). By specifying the depth of puncture needle 3, control section 11 (position specifying section 11b) can specify the transmission/reception directivity characteristic curve of the ultrasound beam which is referred to at the time of specifying the minor axis position of puncture needle 3 as illustrated in FIG. 10A, FIG. 10B and FIG. 10C. It is to be noted that the depth of puncture needle 3 may be determined based on the position of the image of puncture needle 3 with use of a B mode image. In this case, control section 11 (position specifying section 11b) may determine the depth of puncture needle 3 as coordinate data, or as a time period from transmission to reception of an ultrasound beam.

Next, control section 11 (switching control section 11a) outputs a control signal for changing the setting of transducer switching section 24 to register 240 to sequentially deflect the transmission/reception direction of the ultrasound beam on both sides with respect to the center cross-section (minor axis direction), and transmits and receives the ultrasound beam (S3). Then, control section 11 (position specifying section 11b) acquires the reception intensity at the deflection angle of the ultrasound beam at which the reception intensity of the reflection waves from puncture needle 3 is high, and acquires the reception intensity of the reflection waves from puncture needle 3 when the B mode image is acquired (deflection angle: 0 degree) (S4). Thereafter, when the ultrasound beams of the deflection angles of the two directions are transmitted and received, control section 11 (position specifying section 11b) calculates the difference value DIF1 of the detected reception intensities (S5). Then, control section 11 (position specifying section 11b) performs fitting with the transmission/reception directivity characteristic curve of the deflection angles of the two directions based on the difference value DIF1 of the reception intensities of the deflection angles of the two directions as described with FIG. 9B, and specifies the minor axis position of puncture needle 3 at a certain point (S6).

It is to be noted that at the time of specifying the minor axis position with the difference value DIF1 of the reception intensities of the deflection angles of the two directions, the transmission/reception directivity characteristic curve on the graph may not be used, and a method of searching a point where the same numerical value data is obtained may be used as a matter of course. While the difference value DIF1 of the reception intensities expressed by a transmission function are used here, it is also possible to use the difference value DIF1 of the reception intensities expressed by the amplitude of the waveform of the reception signal as it is as a matter of course.

In this manner, control section 11 performs the steps of (S2) to (S6) at a plurality of points along the longitudinal axis direction, and specifies the minor axis position of puncture needle 3 at each point.

Subsequently, control section 11 (detection object specifying section 11c) determines whether the minor axis position of puncture needle 3 is continuous along the longitudinal axis direction (S7). While it is highly possible that intense reflection from the inside of the subject is caused by puncture needle 3, the intense reflection is also caused by the boundary surfaces of the fiber tissues or the like, and the result obtained in (S6) includes the data of such reflections in addition to the data of the puncture needle. To determine whether the data is the data of the puncture needle, when the position of the echo is not continuous along the longitudinal axis direction, it is recognized that the data is not the data of the puncture needle, and the data is dismissed. Then, control section 11 specifies the detection object which is determined to be continuous as puncture needle 3, and outputs the coordinate data representing the minor axis position detected with the reflection waves from puncture needle 3 to image processing section 15 such that image processing section 15 generates a planar image (FIG. 7A) (S8).

FIG. 11 illustrates the method of determining the continuous state in the above-mentioned step (S7). FIG. 11 illustrates original data of the case where the planar image of FIG. 7A is generated. Marks a1 to a10 illustrated in the drawing indicate positions in the minor axis direction which are specified by performing steps (S2) to (S6) at a plurality of points along the longitudinal axis direction. Mark A in the drawing is an object detected at the position a4 in the longitudinal axis direction together with puncture needle 3. The ML in the drawing corresponds to center line ML of FIG. 7A, and is the central axis of the ultrasound beam, and, is the position of the center cross-section of the transmission/reception surfaces of transducers 210a to 210c in the minor axis direction.

The positions of marks a1 to a10 are positions in the minor axis direction specified by sequentially performing steps (S2) to (S6) at positions of a2, a3, a4 and so forth from the position corresponding to a1 in the longitudinal axis direction. Since a portion having a high reflectance such as tumor is present inside the subject Q, an object as mark A other than puncture needle 3 is also detected.

In view of this, as illustrated in FIG. 11, control section 11 (detection object specifying section 11c) uses the data of the minor axis position of puncture needle 3 specified at a plurality of points in the longitudinal axis direction, and thus can determine whether the reflector detected at each position is continuous along the longitudinal axis direction. In other words, control section 11 (detection object specifying section 11c) can determine that marks a1 to a10 are detected with the reflection waves of puncture needle 3, and that mark A is detected with the reflection waves of an object other than puncture needle 3.

To be more specific, control section 11 (detection object specifying section 11c) determines that the positions are not continuous when detection positions (minor axis positions) adjacent to each other in the longitudinal axis direction are separated from each other by a predetermined distance (for example, 0.5 mm). Then, on the basis of the continuous state of the minor axis positions along the longitudinal axis direction, control section 11 (detection object specifying section 11c) discriminates the coordinate data representing the minor axis position detected with the reflection waves from object A other than puncture needle 3, from the coordinate data representing the minor axis position detected with the reflection waves of puncture needle 3. In this manner, control section 11 specifies puncture needle 3 as the detection object from among the detected candidates (reflectors) of puncture needle 3. In addition, control section 11 (position specifying section 11b) can specify each position of puncture needle 3 from the root to the tip portion 3a covered by the ultrasound beam.

With the planar image representing the position of puncture needle 3 generated in the above-mentioned manner, the operator can insert puncture needle 3 toward target G inside the subject Q while determining the orientation and the degree of the deflection of tip portion 3a of puncture needle 3 in the minor axis direction. It is to be noted that image processing section 15 can generate a planar image based on preliminarily prepared image format data of puncture needle 3 and the position of puncture needle 3 specified as described above.

As described above, with the ultrasound diagnostic apparatus according to the present embodiment, the position of the detection object in the minor axis direction can be specified with a higher accuracy by referring to the reception signal of the reflection waves of a detection object such as a puncture needle in the case where the ultrasound beams of the two directions (which include deflection angle of 0 degree) deflected with respect to the minor axis direction are used. Further, the position of a detection object can be specified as a position deflected from the central axis of the ultrasound beam, and therefore the ultrasound diagnostic apparatus according to the present embodiment can be favorably used for generation of a B mode image and the like.

In particular, since the ultrasound diagnostic apparatus specifies the position of the detection object in the minor axis direction with use of the difference value of the reception intensities of the reflection waves of the deflection angles of the two directions, errors due to the gentle directivity of the ultrasound beam can be reduced, and the resolution of the measurement of the position of the detection object in the minor axis direction can be improved.

Additionally, since the ultrasound diagnostic apparatus determines whether the object is puncture needle 3 or a body tissue based on the continuous state of the minor axis position of puncture needle 3 in the longitudinal axis direction, each position of puncture needle 3 from the root to tip portion 3a covered by the ultrasound beam can be specified with high accuracy, and thus a situation where a moving body tissue or the like is erroneously detected as puncture needle 3 can be prevented.

While both the B mode image and the planar image are displayed in the above-mentioned embodiment, the display mode may be appropriately changed. For example, in the case where puncture needle 3 is deflected in the minor axis direction, the display may be changed such that the deflection state can be identified in the B mode image with the color and the type of the line. In addition, the deflection state of puncture needle 3 may be simply displayed with letters or labels.

### (Modification of First Embodiment)

While the reception signals of ultrasound beams of the deflection angles of the two directions are used to specify the minor axis position of a detection object in the above-mentioned embodiment, a reception signal of an ultrasound beam of a deflection angle of another direction may also be used. Also in this case, as with the above-mentioned case, the minor axis position of a detection object can be determined by determining the reception intensities and the difference value obtained with ultrasound beams of different deflection angles, and by searching the minor axis position (abscissa) where the corresponding difference value (ordinate) of the reception intensities of the transmission/reception directivity characteristic curves is obtained.

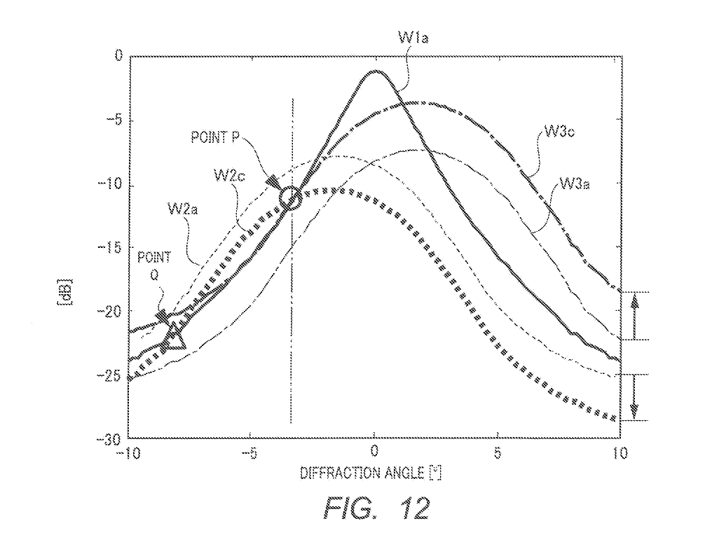

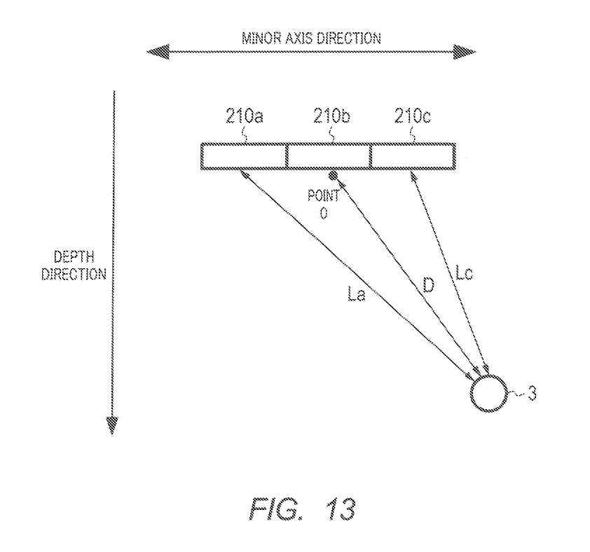

FIG. 12 is an explanatory view of a method of specifying the minor axis position of puncture needle 3 based on the reception intensity obtained using a third ultrasound beam in addition to the first ultrasound beam and the second ultrasound beam. FIG. 12 corresponds to FIG. 9B.

Dotted line W2c in FIG. 12 is a line obtained by lowering the original transmission/reception directivity characteristic curve W2a of the second ultrasound beam as a whole by substantially 1 dB. In addition, dashed line W3c in FIG. 12 is a line obtained by raising the original transmission/reception directivity characteristic curve W3a of the third ultrasound beam as a whole by substantially 5 dB. Here, for convenience of description, it is assumed that the reception intensity from puncture needle 3 detected with use of the first ultrasound beam is −10 dB, the reception intensity from puncture needle 3 detected with use of the second ultrasound beam is −9 dB, and the reception intensity from puncture needle 3 detected with use of the third ultrasound beam is −15 dB.

First, control section 11 (position specifying section 11b) lowers, by the difference value of 1 dB between the reception intensity of −10 dB obtained using the first ultrasound beam and the reception intensity of −9 dB obtained using the second ultrasound beam, the original transmission/reception directivity characteristic curve of the second ultrasound beam (dotted line W2a) as a whole to obtain dotted line W2c. In this case, as illustrated in FIG. 12, solid line W1a and dotted line W2c cross each other at point P and point Q. That is, it is impossible to specify whether point P or point Q is the position of the minor axis position of puncture needle 3 only with the reception intensity of puncture needle 3 detected with use of the first ultrasound beam and the reception intensity of puncture needle 3 detected with use of the second ultrasound beam.

In view of this, control section 11 (position specifying section 11b) uses the reception intensity of puncture needle 3 detected with use of the third ultrasound beam (dashed line W3c) to specify whether point P or point Q is the position of the minor axis position of puncture needle 3.

At this time, control section 11 (position specifying section 11b) raises, by the difference value of 5 dB between the reception intensity of −10 dB obtained using the first ultrasound beam and the reception intensity of −15 dB obtained using the third ultrasound beam, the original transmission/reception directivity characteristic curve of the third ultrasound beam (dotted line W3a) as a whole to obtain dashed line W3c. In this manner, the control section (position specifying section 11b) can specify which of point P and point Q where solid line W1a and dotted line W2c cross each other crosses dashed line W3c. Here, since it is detected that dashed line W3c crosses point P, it is possible to specify that the minor axis position of puncture needle 3 is on the point P side. That is, it is possible to specify that the minor axis position of puncture needle 3 is the position of about −3 degrees.

As described above, in the case where the minor axis position of puncture needle 3 cannot be uniquely specified even when the reception signals of ultrasound beams of the deflection angles of the two directions are used, it is desirable to use a reception signal of an ultrasound beam of a deflection angle of another direction. In other words, with use of reception signals of ultrasound beams of deflection angles of three or more directions, the resolution of the measurement of the position of the detection object in the minor axis direction can be further improved.

### Second Embodiment

Ultrasound diagnostic apparatus U of the present embodiment is different from that of the first embodiment in that the time difference (phase difference) of the reception signals of ultrasound beams of the deflection angles of the two directions are used specify the minor axis position of puncture needle 3. It is to be noted that descriptions for the configurations identical to those of the first embodiment will be omitted (the same applies to the other embodiments).

FIG. 13 is an explanatory view of a method of specifying the minor axis position of puncture needle 3. FIG. 13 illustrates a positional relationship between transducers 210a, 210b and 210c in the minor axis direction, and puncture needle 3 as a reflector at a certain point of. It is to be noted that acoustic lens 22 is omitted in FIG. 13.

In the positional relationship in the drawing, the distance between puncture needle 3 and transducer 210c is shorter than the distance between puncture needle 3 and transducer 210a. Accordingly, for example, the time period from transmission of the ultrasound beam until generation of reflection waves in the case where an ultrasound beam is transmitted and received using transducer 210a is longer than the time period from transmission of the ultrasound beam until generation of reflection waves in the case where an ultrasound beam is transmitted and received using transducer 210c. Here, since the propagation speed of ultrasound waves inside the subject Q can be determined in advance, distances La and Lc between the transmission/reception surfaces of transducers 210a and 210c and a certain point of puncture needle 3 can be determined from the travelling time from transmission of an ultrasound beam until reception after reflection by puncture needle 3. In addition, the positional relationship between transducer 210a and transducer 210c is preliminarily known, and depths D of puncture needle 3 from the center point 0 of the transmission/reception surfaces of transducers 210a to 210c are determined at the time of generation of the B mode image as in the first embodiment.

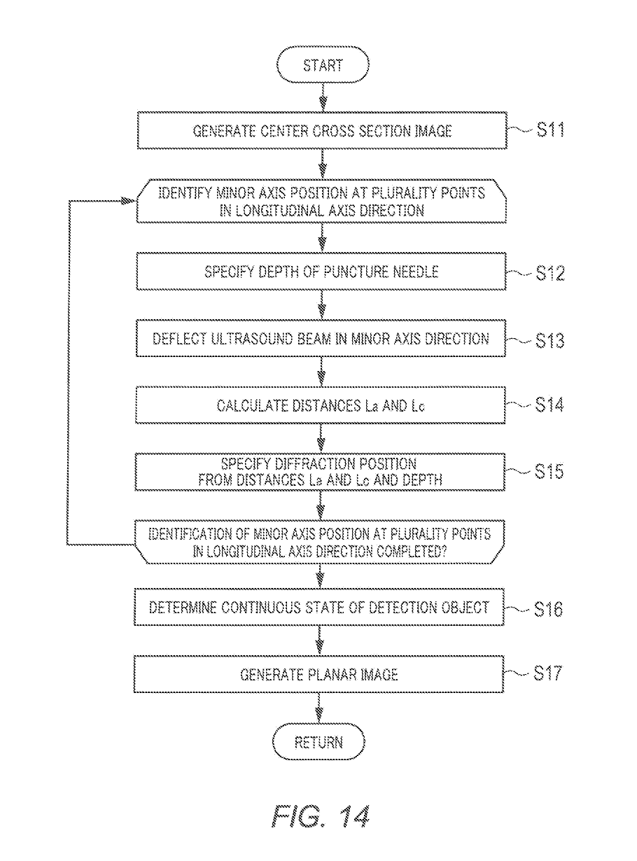

FIG. 14 illustrates an example of a control procedure executed by control section 11. Details of the flow of the control are as follows. First, the control section (position specifying section 11b) specifies center point 0 and the depth D of the object point where the minor axis position of puncture needle 3 is determined based on the temporal variation of the reception signal at the time when the B mode image is acquired (S12). Next, control section 11 (switching control section 11a) outputs a control signal for changing the setting of transducer switching section 24 to register 240, and drives only transducer 210a for transmission and reception of the ultrasound beam, while driving only transducer 210c for transmission and reception of the ultrasound beam (S13). Then, control section 11 (position specifying section 11b) calculates distance La based on the temporal variation of the case where only transducer 210a is driven, and calculates distance Lc based on the temporal variation of the case where only transducer 210c is driven (S14). Thereafter, control section 11 (position specifying section 11b) calculates the distance between center point 0 and point B in the minor axis direction based on depth D, distance La and distance Lc (S15). Thereafter, as with the first embodiment, the minor axis positions at a plurality of points in the longitudinal axis direction are specified, and the continuous state of the detection object is determined (S16), thus generating a planar image (S17).

In the above-mentioned manner, the control section (position specifying section 11b) can calculate the minor axis position of puncture needle 3 with use of distances La and Lc and depth D. Here, it is more desirable to use the phase difference of the reception signals obtained by phase detection at the time of determining distances La and Lc. In this manner, even when the waveform of the reflection waves is not sharp and the arrival time of the reception signal cannot be clearly determined, distances La and Lc can be correctly determined.

As described above, as in ultrasound diagnostic apparatus U according to the present embodiment, also by referring to the reception signal of the reflection waves from the detection object such as a puncture needle or the like in the case where the ultrasound beam deflected in two directions with respect to the minor axis direction (including deflection angle of 0 degree) is used, the position of the detection object in the minor axis direction can be specified with a higher accuracy.

### Third Embodiment

An ultrasound diagnostic apparatus U of the present embodiment is different from that of the first embodiment in that notification section 18 is provided as a configuration for indicating the minor axis position of puncture needle 3 to the operator.

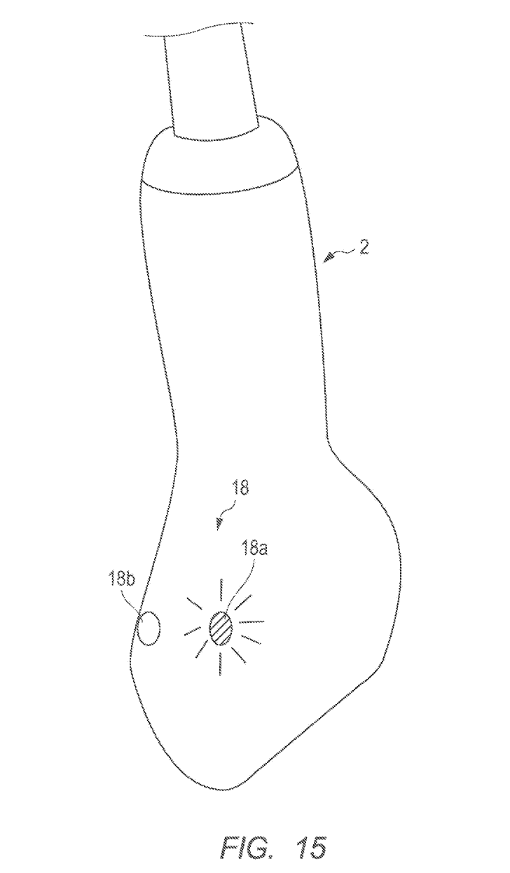

FIG. 15 illustrates an example of notification section 18 for indicating the minor axis position of puncture needle 3.

As with the planar image illustrated in FIG. 7A, notification section 18 operates such that the operator can grasp the deflection side of tip portion 3a of puncture needle 3 in the minor axis. For example, as illustrated in FIG. 15, notification section 18 includes an LED driving circuit (not illustrated) and red and blue LED lamps 18a and 18b which are provided in ultrasound probe 2. In notification section 18, the LED driving circuit is controlled to operate LED lamps 18a and 18b with a control signal from control section 11 (second output control section 11d). In the drawing, a mode in which one of LED lamps 18a and 18b on the deflected direction blinks when tip portion 3a of puncture needle 3 in the minor axis direction is deflected from the center.

In addition, the indication mode of notification section 18 may be changed in accordance with the minor axis position of tip portion 3a of puncture needle 3. For example, notification section 18 operates such that, the blink cycle is shortened as the degree of deflection of tip portion 3a of puncture needle 3 in the minor axis direction from the center position increases, and the blink cycle is lengthened as the degree of the deflection decreases, and, when there is no deflection, (the center position in the minor axis direction), the both LEDs are turned on. In this case, it suffices that control section 11 (second output control section 11d) controls the LED driving circuit in accordance with the specified minor axis position of puncture needle 3.

As described above, with the ultrasound diagnostic apparatus U according to the present embodiment, the operator can insert puncture needle 3 toward target G inside the subject Q while determining the orientation and the degree of the deflection of tip portion 3a of puncture needle 3 in the minor axis direction.

### (Modification of Third Embodiment)

In place of the configuration of indicating the minor axis position of puncture needle 3 with the LED lamps, notification section 18 may perform the indication with a sound. In this case, it suffices that notification section 18 includes a speaker and a speaker driving circuit, and control section 11 (third output of control section 11d) controls the speaker driving circuit (not illustrated) in accordance with the specified minor axis position of puncture needle 3.

As a mode of indication with a sound from a speaker, the deflection side of tip portion 3a of puncture needle 3 in the minor axis direction is indicated to the operator. For example, a sound is output in the direction of the displacement of tip portion 3a of puncture needle 3, or two different sounds, a sound having a high frequency (for example, a sound “peee”) and a sound having a low frequency (for example, a sound “booo”), are output. In addition, notification section 18 outputs an intermittent sound such that the frequently is increased as the degree of deflection from the center position in the minor axis direction increases, and the frequency is decreased as the degree of the deflection decreases, and, the sound is turned off or changed to a continuous sound when there is no deflection (the center position in the minor axis direction).

With this configuration, the operator can insert puncture needle 3 toward target G inside the subject Q while determining the orientation and the degree of the deflection of tip portion 3a of puncture needle 3 in the minor axis direction.

In addition, notification section 18 may indicate the distance between puncture needle 3 and target G to the operator. In this case, control section 11 (third output of control section 11d) specifies the position of target G based on a B mode image and the like, and changes the indication mode depending on the distance between tip portion 3a of puncture needle 3 and target G, for example. Then, for example, notification section 18 starts to output an intermittent sound when tip portion 3a of puncture needle 3 is close to target G, and reduces the interval thereof as the distance decreases, and, outputs a continuous sound when tip portion 3a of puncture needle 3 reaches target G.

With this configuration, the operator can insert puncture needle 3 while confirming the distance of tip portion 3a of puncture needle 3 to target G inside the subject Q.

### Other Embodiments

The present invention is not limited to the above-mentioned embodiment, and may be variously modified.

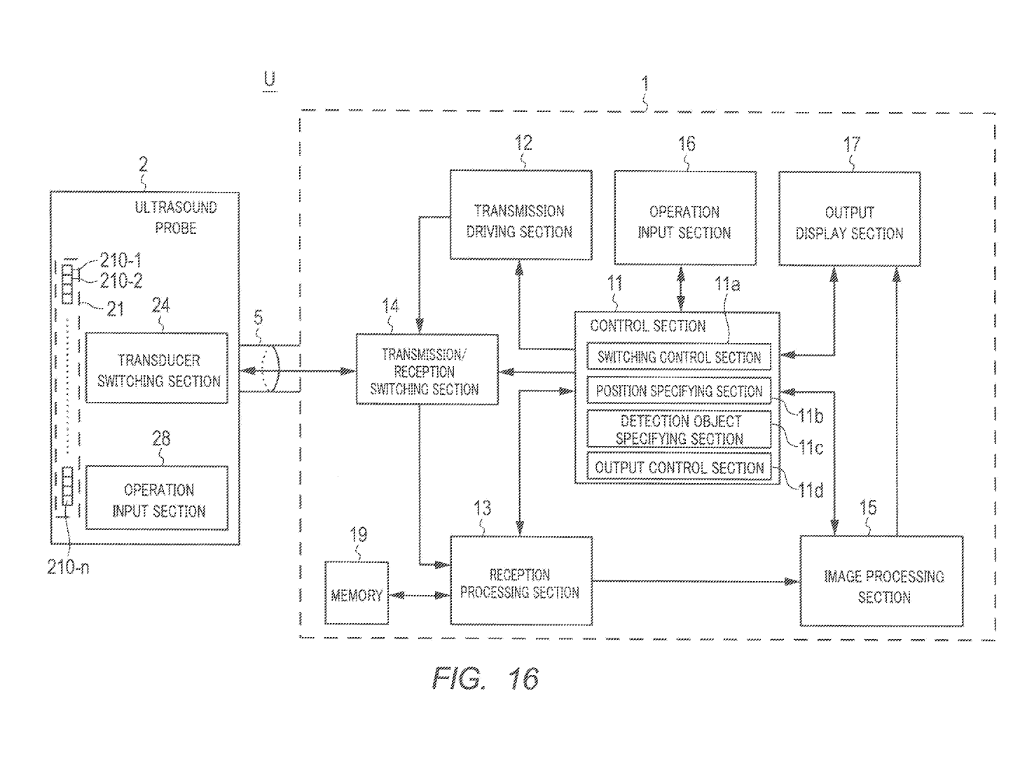

In the above-mentioned embodiment, an exemplary configuration of transducer switching section 24 is described in which the transmission/reception direction of the ultrasound beam is deflected by stopping the operation of transmission and reception of a transducer 210 selected from among the transducer group of 210a to 210c in the minor axis direction. Alternatively, the transmission/reception direction may be deflected with the configuration illustrated in FIG. 16 instead of the above-mentioned configuration. FIG. 16 illustrates a configuration provided with memory 19 in the configuration illustrated in FIG. 5. For example, transmission and reception of the ultrasound beam is performed with use of the line of transducer 210a in the minor axis direction (first transducer group), and the data obtained by the phasing addition process of the phasing addition section (phasing addition circuit) of reception processing section 13 is stored in memory 19. Next, transmission and reception of the ultrasound beam is performed with use of the line of transducer 210b (second transducer group) in the longitudinal axis direction with respect to the same sound ray in the same manner, and the data obtained by the phasing addition process of the phasing addition section (phasing addition circuit) of reception processing section 13 is added to the data stored in memory 19 and the resulting data is stored in memory 19. Reception processing section 13 sends the data generated in the above-mentioned manner from memory 19 to image processing section 15. In this manner, by shifting the timing by the degree of the deflection at the time of addition of the data in memory 19, deflection at a suitable angle can be achieved with the added signal. Likewise, it is also possible to perform addition in consideration of deflection of line a, line b and line c by storing the data of two sound rays in memory 19.

In addition, in the above-mentioned embodiment, transducers 210 of transducer array 21 are disposed in two-dimensional plane in the longitudinal axis direction and the minor axis direction in a matrix as an example configuration of a plurality of transducers 210. However, the configuration of transducers 210 may be appropriately changed, and for example, the portion where transducers 210 are disposed may have a convex shape or the like such that the ultrasound beam is radially transmitted from transducers 210. Alternatively, transducers 210 may be arranged on a concave-convex surface. In this case, the directions of transmission of the ultrasound beams are different among transducers 210, and therefore the ultrasound beam can be deflected by the driven transducers 210 without providing acoustic lens 22 on the transmission/reception surfaces of transducers 210.

In addition, in the above-mentioned embodiment, an example configuration for the operation (computation) for specifying the position or the like of a detection object, one control apparatus 11 includes position specifying section 11b, position specifying section 11b and detection object specifying section 11c. However, the configuration for the operation may not be one component, and the configuration for the operation may be a plurality of components. For example, image processing section 15 may specify the depth of puncture needle 3 based on the image data of the B mode image (at position specifying section 11b).

The disclosure of the specification and drawings includes at least the following matters.

Disclosed is an ultrasound diagnostic apparatus that receives a reception signal representing reflection waves from an ultrasound probe 2, the ultrasound probe 2 including: a plurality of transducers 210 disposed along a first direction (for example, minor axis direction) and configured to transmit an ultrasound beam to a subject Q and receive reflection waves thereof, and a transducer switching section configured to control a driving signal to the transducers 210 to deflect a transmission direction of the ultrasound beam to the first direction, the ultrasound diagnostic apparatus including: a first position specifying section configured to specify a depth of the detection object 3 inside the subject Q based on a temporal variation of the reception signal; a second position specifying section configured to specify a position of the detection object 3 in the first direction based on reception signals generated based on reflection waves from the detection object 3 obtained with the ultrasound beams transmitted at a first angle and a second angle which are different from each other in a transmission direction of the ultrasound beam; and an output control section configured to output the position of the detection object 3 in the first direction such that an operator of the ultrasound probe 2 is allowed to identify the position of the detection object 3 in the first direction. With the ultrasound diagnostic apparatus, the position of a detection object in the first direction can be specified with a higher accuracy.

Preferably, the second position specifying section refers to transmission/reception directivity characteristic data representing a relationship between the position of the detection object 3 in the first direction and an intensity of the reception signal generated based on reflection waves from the detection object 3 which is present at the depth, the transmission/reception directivity characteristic data assuming a case where the ultrasound beams are transmitted and received under a measurement condition where the transmission directions of the ultrasound beams are at the first angle and the second angle, to specify the position of the detection object 3 in the first direction based on the transmission/reception directivity characteristic data, and a difference value of intensities of the reception signals generated based on the reflection waves from the detection object 3 when the transmission directions of the ultrasound beams are at the first angle and the second angle. With the ultrasound diagnostic apparatus, errors due to measurement environments can be reduced, and the resolution of the measurement of the position of a detection object in the first direction can be improved.

Preferably, the second position specifying section refers to transmission/reception directivity characteristic data representing a relationship between the position of the detection object 3 in the first direction and an intensity of the reception signal generated based on reflection waves from the detection object 3 which is present at the depth, the transmission/reception directivity characteristic data assuming a case where the ultrasound beam is transmitted and received under a measurement condition where the transmission direction of the ultrasound beam is at a third angle, to specify the position of the detection object 3 in the first direction based on the transmission/reception directivity characteristic data, and intensities of the reception signals generated based on the reflection waves from the detection object 3 when the transmission directions of the ultrasound beams are at the first angle, the second angle, and the third angle. With the ultrasound diagnostic apparatus, the resolution of the measurement of the position of a detection object in the first direction can be further improved.

Preferably, the second position specifying section specifies the position of the detection object 3 in the first direction as a position deflected from the central axis of the ultrasound beam transmitted from the transducers 210.

Preferably, the ultrasound probe 2 further includes a plurality of transducers 210 disposed along a second direction which crosses the first direction and configured to transmit an ultrasound beam to the subject Q and receive reflection waves thereof, the first position specifying section specifies a depth of the detection object 3 at a plurality of points along the second direction based on a temporal variation of reception signals of the transducers 210 disposed along the second direction, and the second position specifying section specifies the position of the detection object 3 in the first direction at the plurality of points along the second direction. With the ultrasound diagnostic apparatus, positions to the tip portion of the detection object in the first direction can be specified.

Preferably, the detection object 3 is a puncture needle, the second direction is a direction along which the puncture needle is inserted, and the first direction is a direction orthogonal to the direction along which the puncture needle is inserted. With the ultrasound diagnostic apparatus, positions to the end of the tip portion of the puncture needle in the first direction can be specified.

Preferably, the ultrasound diagnostic apparatus further includes a detection object 3 specifying section configured to specify a type of the detection object 3 based on a continuous state of positions of the detection object 3 in the first direction specified at the plurality of points along the second direction. With the ultrasound diagnostic apparatus, a reflector other than the detection object can be discriminated from the detection object, and positions to the end of the tip portion of the detection object in the first direction can be specified.

Preferably, the output control section operates to display an image representing the position of the detection object 3 in the first direction such that the image corresponds to a tomographic image of the subject Q generated along the second direction based on the position of the detection object 3 in the first direction specified at the plurality of points along the second direction. With the ultrasound diagnostic apparatus, the operator can insert puncture needle 3 toward target G inside the subject Q while determining the orientation and the degree of the deflection of tip portion 3a of puncture needle 3 in the minor axis direction.

Preferably, the output control section indicates the position of the detection object 3 in the first direction to the operator of the ultrasound probe 2 in an identification mode corresponding to the position of the detection object 3 in the first direction. With the ultrasound diagnostic apparatus, the operator can insert puncture needle 3 toward target G inside the subject Q while determining the orientation and the degree of the deflection of tip portion 3a of puncture needle 3 in the minor axis direction.

Preferably, the ultrasound diagnostic apparatus further includes: a phasing addition section configured to perform phasing addition of reception signals obtained from the transducers 210; a memory configured to temporarily store reception data subjected to first phasing addition obtained from a reception signal acquired by a first transducer group disposed along a line in the first direction; and a memory configured to add the reception data subjected to the first phasing addition stored in the memory to reception data subjected to second phasing addition obtained from a reception signal acquired by a second transducer group disposed along a line different from that of the first transducer group in the first direction. Transmission of the ultrasound beam is performed a plurality of times with respect to the same sound ray in a longitudinal axis direction to perform deflection control.

Disclosed is a control method for an ultrasound diagnostic apparatus that receives a reception signal representing reflection waves from an ultrasound probe 2, the ultrasound probe 2 including: a plurality of transducers 210 disposed along a first direction and configured to transmit an ultrasound beam to a subject Q and receive reflection waves thereof, and a transducer switching section configured to control a driving signal to the transducers 210 to deflect a transmission direction of the ultrasound beam to the first direction, the method including: specifying a depth of the detection object 3 inside the subject Q based on a temporal variation of the reception signal; specifying a position of the detection object 3 in the first direction based on reception signals generated based on reflection waves from the detection object 3 obtained with the ultrasound beams transmitted at a first angle and a second angle which are different from each other in a transmission direction of the ultrasound beam; and outputting the position of the detection object 3 in the first direction such that an operator of the ultrasound probe 2 is allowed to identify the position of the detection object 3 in the first direction.

It should be understood by those skilled in the art that various modifications, combinations, sub-combinations and alterations may occur depending on design requirements and other factors in so far as they are within the scope of the appended claims or the equivalents thereof.

What is claimed is:

1. An ultrasound diagnostic apparatus that receives a reception signal representing reflection waves from an ultrasound probe, the ultrasound probe including:
a plurality of transducers disposed along a first direction and configured to transmit an ultrasound beam to a subject and receive reflection waves thereof, and
a transducer switching section configured to control a driving signal to the transducers to deflect a transmission direction of the ultrasound beam to the first direction, the ultrasound diagnostic apparatus comprising:
a first position specifying section configured to specify a depth of a detection object inside the subject based on a temporal variation of the reception signal;
a second position specifying section configured to specify a position of the detection object in the first direction based on reception signals generated based on reflection waves from the detection object obtained with the ultrasound beams transmitted at a first angle and a second angle which are different from each other in a transmission direction of the ultrasound beam; and
an output control section configured to output the position of the detection object in the first direction such that an operator of the ultrasound probe is allowed to identify the position of the detection object in the first direction.

2. The ultrasound diagnostic apparatus according to claim 1, wherein
the second position specifying section refers to transmission/reception directivity characteristic data representing a relationship between the position of the detection object in the first direction and an intensity of the reception signal generated based on reflection waves from the detection object which is present at the depth, the transmission/reception directivity characteristic data assuming a case where the ultrasound beams are transmitted and received under a measurement condition where the transmission directions of the ultrasound beams are at the first angle and the second angle,
to specify the position of the detection object in the first direction based on the transmission/reception directivity characteristic data, and a difference value of intensities of the reception signals generated based on the reflection waves from the detection object when the transmission directions of the ultrasound beams are at the first angle and the second angle.

3. The ultrasound diagnostic apparatus according to claim 2, wherein
the second position specifying section refers to transmission/reception directivity characteristic data representing a relationship between the position of the detection object in the first direction and an intensity of the reception signal generated based on reflection waves from the detection object which is present at the depth, the transmission/reception directivity characteristic data assuming a case where the ultrasound beam is transmitted and received under a measurement condition where the transmission direction of the ultrasound beam is at a third angle,
to specify the position of the detection object in the first direction based on the transmission/reception directivity characteristic data, and intensities of the reception signals generated based on the reflection waves from the detection object when the transmission directions of the ultrasound beams are at the first angle, the second angle, and the third angle.

4. The ultrasound diagnostic apparatus according to claim 1, wherein the second position specifying section specifies the position of the detection object in the first direction as a position deflected from the central axis of the ultrasound beam transmitted from the transducers.

5. The ultrasound diagnostic apparatus according to claim 1, wherein
the ultrasound probe further includes a plurality of transducers disposed along a second direction which crosses the first direction and configured to transmit an ultrasound beam to the subject and receive reflection waves thereof,
the first position specifying section specifies a depth of the detection object at a plurality of points along the second direction based on a temporal variation of reception signals of the transducers disposed along the second direction, and
the second position specifying section specifies the position of the detection object in the first direction at the plurality of points along the second direction.

6. The ultrasound diagnostic apparatus according to claim 5, wherein
the detection object is a puncture needle,
the second direction is a direction along which the puncture needle is inserted, and
the first direction is a direction orthogonal to the direction along which the puncture needle is inserted.

7. The ultrasound diagnostic apparatus according to claim 5 further comprising a detection object specifying section configured to specify a type of the detection object based on a continuous state of positions of the detection object in the first direction specified at the plurality of points along the second direction.

8. The ultrasound diagnostic apparatus according to claim 5, wherein the output control section operates to display an image representing the position of the detection object in the first direction such that the image corresponds to a tomographic image of the subject generated along the second direction based on the position of the detection object in the first direction specified at the plurality of points along the second direction.

9. The ultrasound diagnostic apparatus according to claim 1, wherein the output control section indicates the position of the detection object in the first direction to the operator of the ultrasound probe in an identification mode corresponding to the position of the detection object in the first direction.

10. The ultrasound diagnostic apparatus according to claim 2 further comprising:
a phasing addition section configured to perform phasing addition of reception signals obtained from the transducers;
a memory configured to temporarily store reception data subjected to first phasing addition obtained from a reception signal acquired by a first transducer group disposed along a line in the first direction; and
a memory configured to add the reception data subjected to the first phasing addition stored in the memory to reception data subjected to second phasing addition obtained from a reception signal acquired by a second transducer group disposed along a line different from that of the first transducer group in the first direction, wherein
transmission of the ultrasound beam is performed a plurality of times with respect to the same sound ray in a longitudinal axis direction to perform deflection control.

11. A control method for an ultrasound diagnostic apparatus that receives a reception signal representing reflection waves from an ultrasound probe, the ultrasound probe including:
a plurality of transducers disposed along a first direction and configured to transmit an ultrasound beam to a subject and receive reflection waves thereof, and
a transducer switching section configured to control a driving signal to the transducers to deflect a transmission direction of the ultrasound beam to the first direction, the method comprising:
specifying a depth of the detection object inside the subject based on a temporal variation of the reception signal;
specifying a position of the detection object in the first direction based on reception signals generated based on reflection waves from the detection object obtained with the ultrasound beams transmitted at a first angle and a second angle which are different from each other in a transmission direction of the ultrasound beam; and
outputting the position of the detection object in the first direction such that an operator of the ultrasound probe is allowed to identify the position of the detection object in the first direction.
